(19) United States
(12) Patent Application Publication
(10) Pub. No.: US 2005/0210568 A1
(43) Pub. Date: Sep. 29, 2005

(54) Lens attachment combined with formation of goggles frame

(76) Inventor: Brent Sheldon, Montreal (CA)

Correspondence Address: OGILVY RENAULT LLP, 1981 MCGILL COLLEGE AVENUE, SUITE 1600, MONTREAL, QC, H3A2Y3, CA

(21) Appl. No.: 10/809,335

(22) Filed: Mar. 26, 2004

(51) Int. Cl. A61F 9/02

(52) U.S. Cl. 2/426

(57) ABSTRACT

The present invention provides a method and structural arrangement for permanently attaching lenses to goggles which include a shielding member contoured for shielding the eyes of a user. The attachment of the lenses to the frame structures of goggles is achieved during the formation of the frame structures. A variety of engaging structures between the lenses and a frame body can be formed together with the formation of the frame structure in a plastic formation molding procedure.

### FIELD OF THE INVENTION

The present invention relates to goggles, and more particularly to a method and a structural arrangement for attaching lenses to frames of goggles.

### BACKGROUND OF THE INVENTION

Distinct from eye glasses, goggles provide protection for user's eyes not only by the lenses attached thereto but also by seals or other types of shielding members which are integrated with or attached to the frames of goggles and contoured for better shielding user's eyes.

Goggles are generally cataloged into two groups. One group of goggles require a tight seal around the user's eyes to prevent fluids such as water, from contacting the eyes. The goggles of this type include swimming goggles, diving goggles, and the like. The attachment of lenses to the frames of the goggles of this type, are required to be fluid-tight in order to prevent fluid leakage. Therefore, the frames are typically made of flexible and resilient material, for example, soft or semi-rigid plastics or rubber, such that it is convenient to achieve a tight seal of the frames around user's eyes.

The other group of goggles require ventilation in order to circulate air flow between the lenses and the user's eyes, thereby preventing condensation of the lenses. The ventilation passages can be provided in the shielding members, frame bodies or lenses. The goggles of this type are widely used in a variety of outdoor sports, and as protective goggles in some work situations. The attachment of lenses to the goggles of this type are not required to be fluid-tight. The frames are usually made of both soft or semi-rigid and substantially rigid material, depending on the specific configurations thereof. For those goggles having substantially rigid frames, the attachment of the lenses to the frames, similar to that of eye glasses, is usually by a “click-in” action in which the lenses are pressed into apertures of the frames and held in position by forces resulting from elastic deformation of both the lenses and the frame bodies. However, neither the rigid lenses nor the rigid frame bodies defining the apertures provide a significant elastic deformation during the “click in” action for a more secure attachment of the lenses to the frames. Goggles users unfortunately sometimes suffer the loss of lenses from their goggles because the lenses have become less firmly retained within the apertures of the frames over a period of usage. It should be noted that the conventional “click in” type of attachment of lenses to the apertures of the goggles frames requires very accurate geometry of both the peripheries of lenses and the apertures of the frames, when the lenses and frames are fabricated in separate manufacturing processes.

Therefore, there is a need for a secure attachment of lenses to the frames of goggles having ventilation, particularly to the substantially rigid plastic frames of goggles having ventilation.

### SUMMARY OF THE INVENTION

One object of the present invention is to provide a method and a structural arrangement for attaching a lens to a plastic frame of goggles having ventilation.

In accordance with one aspect of the present invention, there is a method provided for attaching a substantially rigid lens to a substantially rigid plastic frame of goggles having ventilation, the frame including a shielding member contoured for shielding the eyes of a user. The method comprises steps of providing a molding device for molding the substantially rigid plastic frame; placing the lens in a predetermined position with respect to the molding device, in the predetermined position a portion of the molding device overlapping a portion of the lens; and forming the substantially rigid plastic frame with permanent engagement of the lens thereto using the molding device.

In accordance with another aspect of the present invention there is a structural arrangement for attaching a lens to a frame having ventilation, the frame including a shielding member contoured for shielding the eyes of a user. The structural arrangement comprises a frame body made of a plastic material, defining an aperture receiving the lens therein and defining a plurality of ventilation passages for circulating air between the face of the user and the lens. A plurality of retaining members are integrally formed together with the frame body. The respective retaining members are contoured to achieve permanent attachment of the lens to the plastic frame body during formation of the frame body.

In accordance with a further aspect of the present invention, there is a structural arrangement provided for attaching a substantially rigid lens to a frame of goggles having ventilation, the frame including a shielding member contoured for shielding the eyes of a user. The structural arrangement comprises a frame body made of a substantially rigid plastic material. A plurality of retaining members are integrally formed together with the substantially rigid plastic frame body. The respective retaining members are configured to achieve permanent attachment of the lens to the substantially rigid plastic frame body during formation of the frame body.

The method and structural arrangement of the present invention provides a permanent attachment of lenses to the frames of goggles having ventilation, which eliminates the possibility of losing lenses from the eye glasses.

Other features and advantages of the present invention will be better understood with reference to the preferred embodiments described hereinafter.

### BRIEF DESCRIPTION OF THE DRAWINGS

Having thus generally described the nature of the present invention, reference will now be made to the accompanying drawings, showing by way of illustration the preferred embodiments thereof, in which:

FIG. 1 is a perspective view of goggles having ventilation, configured for permanent attachment of a lens to a frame structure in accordance with one embodiment of the present invention;

FIG. 2A is a front elevational view of the goggles of FIG. 1, showing retaining members integrally formed with the frame body;

FIG. 2B is a partial cross-sectional view of the goggles of FIG. 2A, taken along line 2-2, showing a structural arrangement of this embodiment;

FIG. 2C is a partial cross-sectional view similar to the view of FIG. 2B, showing a formation molding procedure of the frame body with permanent attachment of the lens thereto;

FIG. 2D is a cross-sectional view of the goggles of FIG. 2A, taken along 2-2 showing an alternative structural arrangement of this embodiment;

FIG. 2E is a partial cross-sectional view of the goggles of FIG. 2A, taken along line 2-2 showing a further alternative structural arrangement thereof;

FIG. 3A is a front elevational view of goggles having ventilation, configured for permanent attachment of a lens to the frame structure thereof according to a second embodiment of the present invention;

FIG. 3B is a partial cross-sectional view of the goggles of FIG. 3A, taken along line 3-3, showing a structural arrangement thereof;

FIG. 3C is a partial cross-sectional view of the eye glasses of FIG. 3A, taken along line 3-3, showing an alternative structural arrangement thereof;

FIG. 4A is a front elevational view of goggles having ventilation, configured for permanent attachment of a lens to the frame structure thereof in accordance with a third embodiment of the present invention;

FIG. 4B is a partial cross-sectional view of the goggles of FIG. 4A, taken along line 4-4, showing a structural arrangement thereof;

FIG. 4C is a partial cross-sectional view similar to that of FIG. 4B, showing an alternative structural arrangement thereof;

FIG. 5 is a perspective view of goggles having ventilation, configured for permanent attachment of a pair of lenses to the frame structure thereof in accordance with a fourth embodiment of the present invention;

FIG. 6A is a front elevational view of the goggles of FIG. 5, showing the frame body thereof;

FIG. 6B is a partial cross-sectional view of the goggles of FIG. 6A, taken along line 6-6, showing a structural arrangement thereof;

FIG. 6C is a partial cross-sectional view of the lens shown in FIG. 6B, showing a hollow space defined therein;

FIG. 6D is a front elevational view of the lens shown in FIG. 6B taken along line 6-6, showing the hollow space defined therein;

FIG. 6E is a front elevational view of the lens shown in FIG. 6B, showing a hollow space defined therein according to an alternative structural arrangement; and

FIG. 7A is a front elevational view of goggles having ventilation, configured for permanent attachment of lenses to the frame structure thereof in accordance with a fifth embodiment of the present invention.

### DETAILED DESCRIPTION OF THE PREFERRED EMBODIMENTS

It should be noted that the term “goggles having ventilation” used throughout the entire specification and appended claims includes a variety of goggles which allow for air ventilation and require neither fluid-tight attachment of lenses to the frames thereof, nor fluid-tight seals around the eyes of users, and therefore, exclude swimming goggles, diving goggles, and the like. It should also be noted that the term “a shielding member contoured for shielding the eyes of a user” used throughout the entire specification and the appended claims, means a structural element attached to or integrated with the frame structure of goggles, substantially as a shielding feature additional to the shielding feature of the lenses of the goggles.

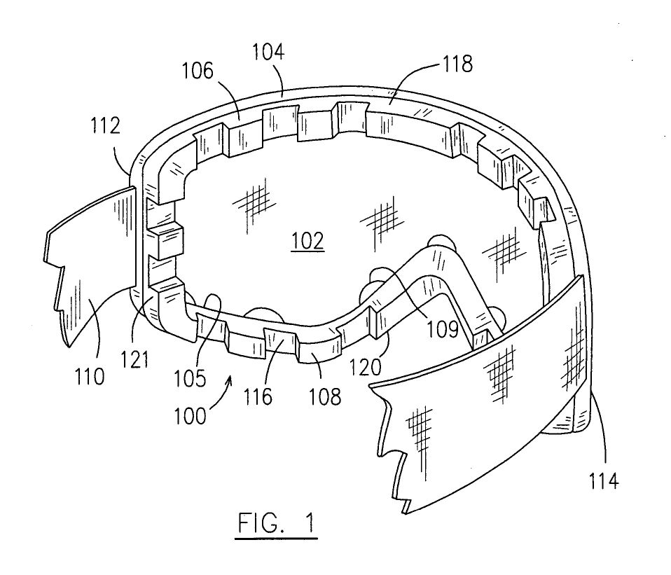

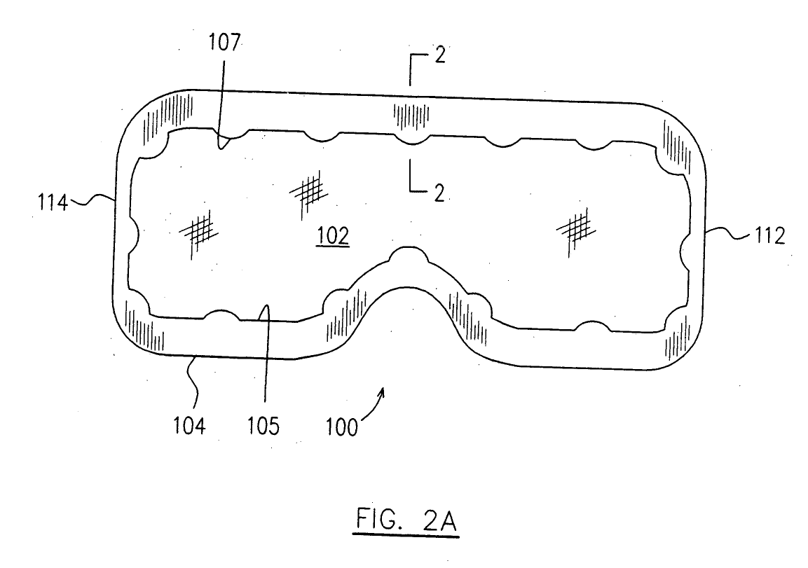

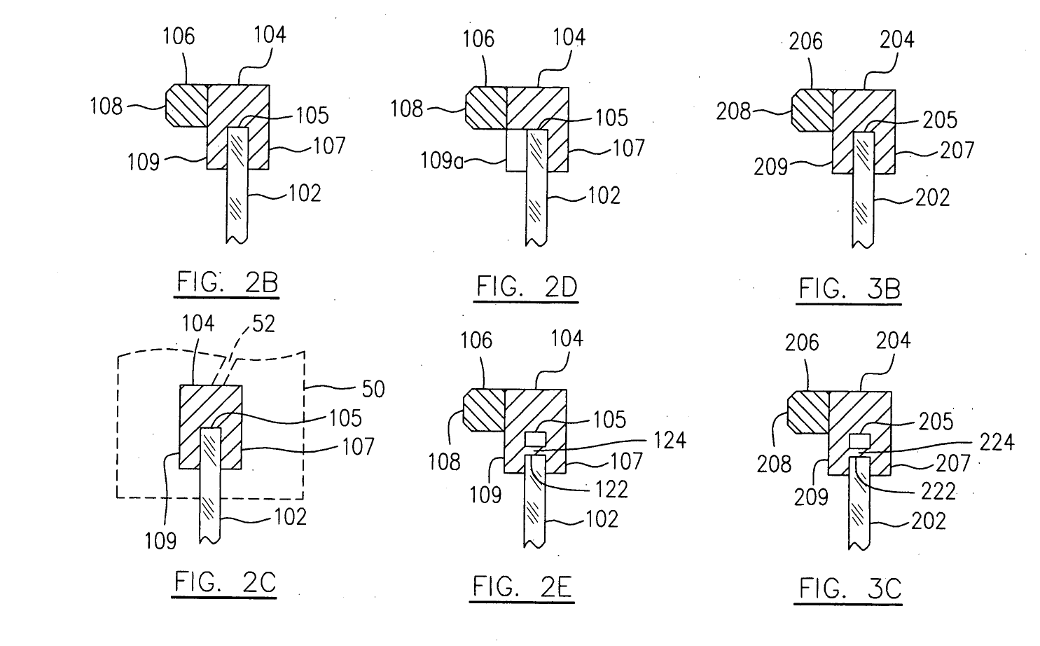

FIGS. 1 and 2A-2E illustrate goggles having ventilation such as protective goggles, indicated by numeral 100, in accordance with a first embodiment of the present invention. The goggles 100 is used as an example to illustrate one embodiment of the present invention. Nevertheless, any other type of goggles which includes substantially rigid plastic frame bodies and substantially rigid lenses, and which defines ventilation passages therewith in various ways, is included in the concept of the present invention as illustrated by the embodiment 100 and in further embodiments to be described hereinafter.

The goggles 100 includes a single lens 102 which is made of glass or substantially rigid transparent plastic, a frame body 104 defining an aperture 105 receiving the single lens 102 therein, and a shielding member 106 contoured for shielding the eyes of a user. The frame body 104 is made of a substantially rigid plastic material and includes a plurality of retaining members 107 and 109 for securing the lens 102. The shielding member 106 which is preferably made of a semi-rigid material such as rubber in this embodiment, is attached to the inner side of the frame body 104 and has a contacting surface 108. The combination of the single lens 102, the frame body 104 and the shielding member 106, is contoured such that the contacting surface 108 of the shielding member 106 contacts the face of the user (not shown) in a comfortable manner and the shielding member thereby forms a substantial enclosure between the single lens 102 and the face of the user when the goggles 100 is worn.

An elastic strap 110 is attached to the opposite sides 112, 114 of the frame body for holding the goggles 100 on the head of the user by a means well known in the art which will therefore not be described herein.

A plurality of ventilation channels 116 are formed on the contacting surface of the shielding member 106 in the top section 118 and bottom section 120 as well as side sections 121, such that air circulation through the enclosure defined between the single lens 102 and the face of the user eliminate condensation on the single lens 102.

The retaining members 107 which are referred as the retaining members of a first group hereinafter, are integrally formed together with the frame body 104 and extend into the aperture 105. The retaining members 109 which are referred to as retaining members of a second group hereinafter, are also integrally formed together with the frame body 104 and extend into the aperture 105. The second group of retaining members 109 is spaced apart from the first group of retaining members 107, and the first and second groups of retraining members 107, 109 abut the respective opposed front and rear sides of the single lens 102, thereby engaging a plurality of peripheral parts of the single lens 102 therebetween. The number and size of the first and second groups of the retaining members 130, 132 may be identical and may be disposed to correspond one with another in the circumferential direction of the aperture 105, as shown in FIG. 2B, but this is optional rather than necessary. The first and second groups of the retaining members 107, 109 must have a dimension thereof which is much greater than the total allowed maximum elastic deformation of the single lens 102 and the frame body 104, in order to prevent disengagement of the single lens 102 from the first and second groups of the retaining members 107, 109, thereby ensuring permanent attachment of the single lens 102 within the aperture 105.

In FIG. 2C there is an illustration of a frame body formation molding procedure together with the attachment of the single lens 102 to the frame body 104. A molding device 50 is indicated with a broken line representing a partial section thereof. The molding device 50 defines a cavity (not indicated) in accordance with a configuration of the frame body 104 and the first and second groups of the retaining members 107, 109, and includes a molding injection passage 52 communicating with this cavity in order to permit injection of the plastic material in an appropriate state into the cavity during a formation molding procedure.

The single lens 102 is placed in a predetermined position with respect to the molding device 50 such that at least a portion of the molding device overlaps a portion of the single lens 102, and the single lens 102 closes major openings of the cavity of the molding device 50 except for the molding injection passage 52 and air escape passages (not shown). When the single lens 102 is placed and held by tools (not shown) in this predetermined position and closes the major openings of the cavity of the molding device 50, the formation molding procedure begins and the cavity is filled with the plastic material, thereby forming the substantially rigid plastic frame body 104 which defines the aperture 105 therein, with permanent attachment of the lens thereto.

The shielding member 106 is then attached to the inner side of the frame body 104 by any well known means, such as glue. However, if the shielding member 106 is preferred to be an integral part of the frame body 104, it can be formed together with the frame body 104 during the formation of the latter, provided that the molding device 50 is provided with an appropriate cavity therein.

FIG. 2D illustrates an alternative structural arrangement of the embodiment shown in FIG. 2A. In this alternative structural arrangement, the second group of retaining members which are indicated by 109a to be distinguished from the second group of retaining members 109 of FIG. 2B, are positioned to be not aligned with the first group of retaining members 107, respectively. The first and second groups of the retaining members 107, 109a are offset in the circumferential direction, which does not affect the permanent engagement of the single lens 102 within the frame body 104, provided that the dimension of the first and second groups of retaining members 107, 109a of the structural arrangement of FIG. 2D is not smaller than the dimension of the first and second groups of the retaining members 107, 109 of the structural arrangement of FIG. 2B.

A further alternative structural arrangement of the goggles 100 of FIG. 2A is illustrated in FIG. 2E. The single lens 102 is provided with a hollow space defined therein, for example, a plurality of holes 122 extending through the single lens 102. The holes 122 are disposed in the respective peripheral parts of the single lens 102 corresponding to the respective positions of the aligned first and second groups of the retaining members 107, 109. During the formation molding procedure of the frame body 104, similar to that illustrated in FIG. 2C, a portion of a plastic material which is in an appropriate state to form the frame body 104 is filled into the respective holes 122 defined through the single lens 102, thereby forming a plurality of studs 124 integrated with the frame body 104. Each of the studs 124 extends between one of the first group of retaining members 107 and one of the second group of retaining members 109 through one of the holes 122. These studs 124 reinforce the permanent attachment of the single lens 102 to the frame body 104.

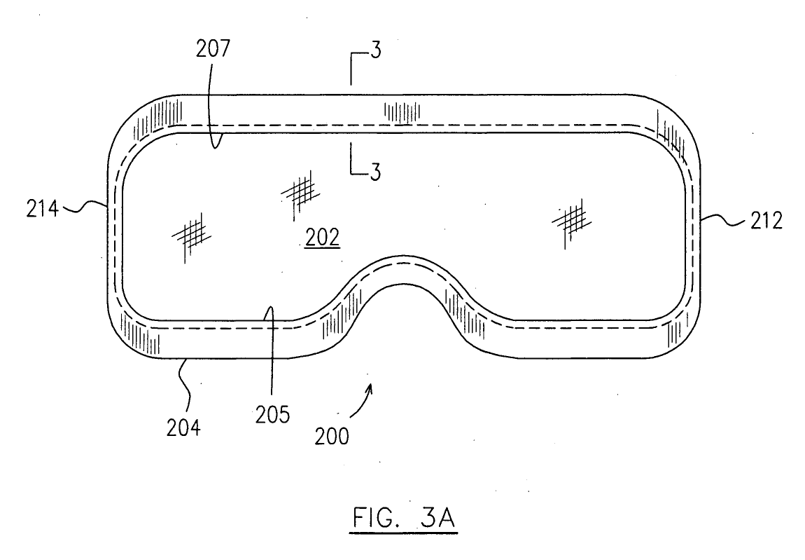

FIGS. 3A-3C illustrate a second embodiment of the present invention in which goggles 200 having ventilation is similar to the goggles 100 of FIGS. 2 and 2A. The components of goggles 200 are indicated using numerals in the 200 series with the last two digits corresponding to similar components in the 100 series illustrated in FIGS. 2 and 2A, and will not therefore be redundantly described. The embodiment shown in FIG. 3A is a further development of the embodiment shown in FIGS. 2 and 2A. When the retaining members 107 and 109 of the eye glasses 100 (as shown in FIGS. 2A and 2B), are connected adjacent to one another in each of the first and second groups, these retaining members substantially form opposed continuous side walls 207, 209 extending into an aperture 205 of the goggles 200, thereby defining a continuous channel therebetween (not indicated). The continuous channel defined between opposed side walls 207, 209 is configured to correspond to the periphery of a single lens 202 for receiving the periphery of the single lens 202, and has a depth which is much greater than the amount of total maximum elastic deformation of the single lens 202 and a frame body 214, in order to permanently engage the single lens 202 when the attachment of the single lens 202 to the aperture 205 is completed during the formation of the frame body 204.

The formation of the frame body 204 is completed in a molding process using a molding device (not shown) similar to the molding device 50 of FIG. 2C with a cavity defined therein corresponding to the configuration of the frame body 204, and particularly to the configuration of the opposed continuous side walls 207, 209. The single lens 202 is placed in a predetermined postiion with respect to the molding device. A continuous portion of the molding device overlaps a continuous periphery portion of the single lens 202 at both sides thereof (the cross-section will be similar to that illustrated in FIG. 2C) such that the frame body 204 is formed with the opposed continuous walls 207, 209 extending therefrom into the aperture 205.

It should be noted that in this embodiment and other embodiments to be further described, the molding devices are similar to the molding device 50 with different cavities defined therein. In order to focus on the structural features of each embodiment of the present invention and avoid redundant illustration, the other molding devices will not be shown and the description of the corresponding formation molding procedures will be referred to the molding device 50 of FIG. 2C.

FIG. 3C illustrates an alternative structural arrangement for the permanent attachment of the single lens 202 to the aperture 205 of the goggles 200. In this alternative structural arrangement, the single lens 202 is provided with a hollow space therein, for example, a plurality of holes 222 extending through a peripheral portion of the single lens 202. During the molding procedure of the frame body 204, the peripheral portion of the lens defining the holes 222 therein, overlaps a portion of a molding device similar to the molding device 50 of FIG. 2C such that a portion of the frame body 204 is filled into the hollow space of the single lens 202, thereby forming a plurality of studs 224 integrated with the frame body 204 and extending between the side walls 207 and 209 through the holes 222. The holes 222 are spaced apart from one another along the periphery of the single lens 202, and thus the studs 224 extending therethrough reinforce the permanent attachment of the single lens 202 to the aperture 205 defined in the frame body 204.

It should be noted that the side walls 207 and 209 in the structural arrangement shown either in FIG. 3B or 3C, may not necessarily be continuous along the circumferential direction, circling the aperture 205. For example, upper and lower sections of the opposed side walls 207 and 209 are sufficient to secure the single lens 202.

The attachment of the lens to the apertures of the frame structure of goggles according to the present invention is achieved by permanent engagement resulting from specific structural arrangements between the lens and the frame body, rather than frictional forces therebetween resulting from elastic deformation of both lens and frame body as in the prior art. Thus, the attachment of the lens to the frame structure according to the present invention is much more secure and completely eliminates the risk of losing the lens. Furthermore, the structural arrangement for the permanent engagement of a lens according to the present invention requires less accurate peripheral geometry of the lens because the attachment of the lens to the aperture of the frame bodies is achieved during the formation of the frame bodies, which results in an automatic match between the periphery of the lens and the inner periphery of the frame body defining the aperture.

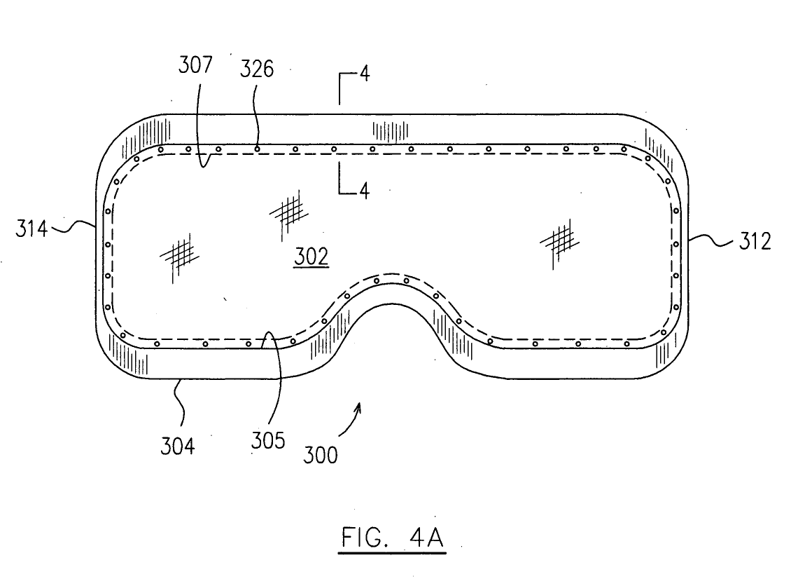

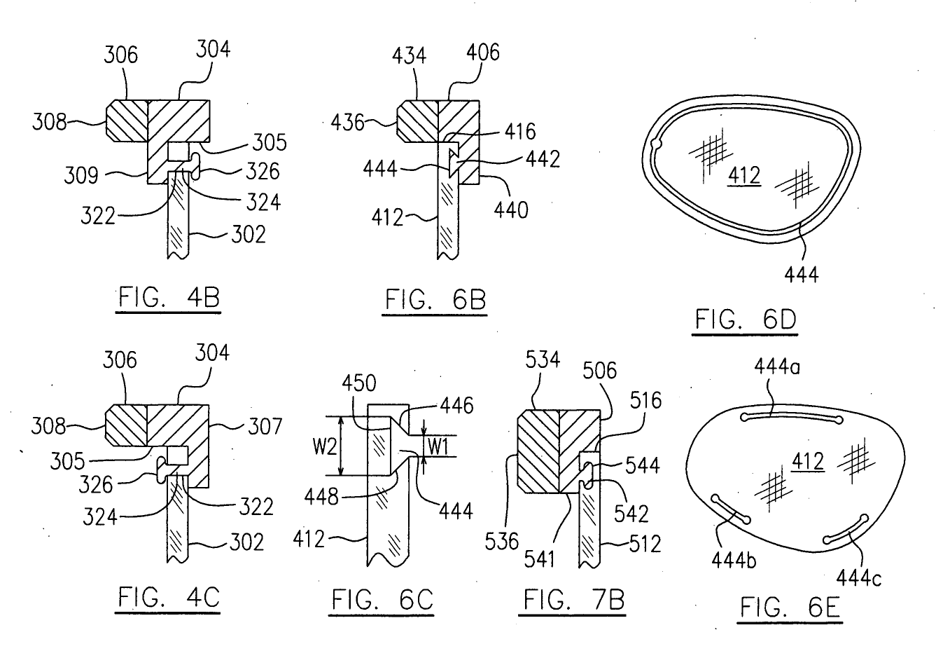

FIGS. 4A-4C illustrate a third embodiment of the present invention generally indicated by numeral 300. The embodiment 300 is goggles having ventilation including components similar to those of the goggles 100 shown in FIGS. 1 and 2A-2E. These similar components are indicated by numerals in the 300 series with the last two digits similar to those corresponding components of the goggles 100 of FIGS. 1 and 2A-2E, and will not therefore be redundantly described. Instead of having the plurality of retaining members 107, 109 of FIG. 2B, the goggles 300 includes a structural arrangement in which a single lens 302 is provided with a hollow space defined as a plurality of holes 322 extending through the single lens 302. The holes 322 are spaced apart one from another and are defined in a peripheral portion of the single lens 302. The peripheral portion of the single lens 302 defining the holes 322 therein abuts a continuous rear side wall 309 extending from a frame body 304 into an aperture 305, defined within the frame body 304. A plurality of studs 324 formed integrally with the frame body 304, are filled into the respective holes 322. Furthermore, the studs 324 extend from the continuous rear side wall 309 through the respective holes 322. Each stud 324 terminates at an enlarged end 326 thereof which has a size greater than the diameter of the stud. Thus the studs 324 permanently engage the single lens 302 in the aperture 305 between the continuous rear side wall 309 and the enlarged end 326 of the studs 324. This structural arrangement is also achieved during the formation of the frame body 304 in a molding procedure similar to that illustrated in FIG. 2C. An appropriate cavity configuration should be provided in the molding device in order to mold the frame body 304 such that when the single lens 302 is placed in the predetermined position in which the single lens 302 overlaps a portion of the molding tool and closes major openings of the cavity, the molding plastic material will fill the cavity, thereby forming the frame body 304 and the studs 324 with their enlarged ends 326 with the attachment of the single lens 302 thereto.

In an alternative arrangement illustrated in FIG. 4C, a continuous side wall can be disposed in the front of the goggles 300 such that the continuous rear side wall 309 of FIG. 4B becomes a continuous front side wall 307 of FIG. 4C, and the plurality of enlarged ends 326 of the studs 324 are disposed behind the single lens 302, resulting in the front view of the eye glasses 300 appearing similar to the goggles 200 illustrated in FIG. 3A.

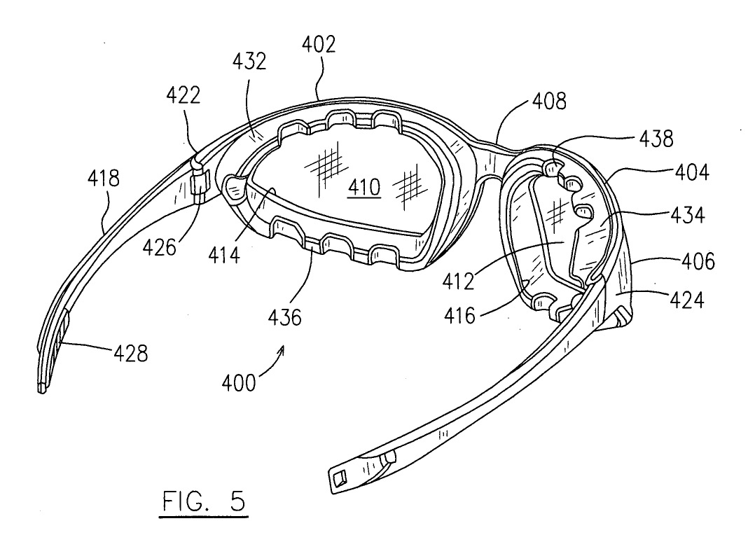

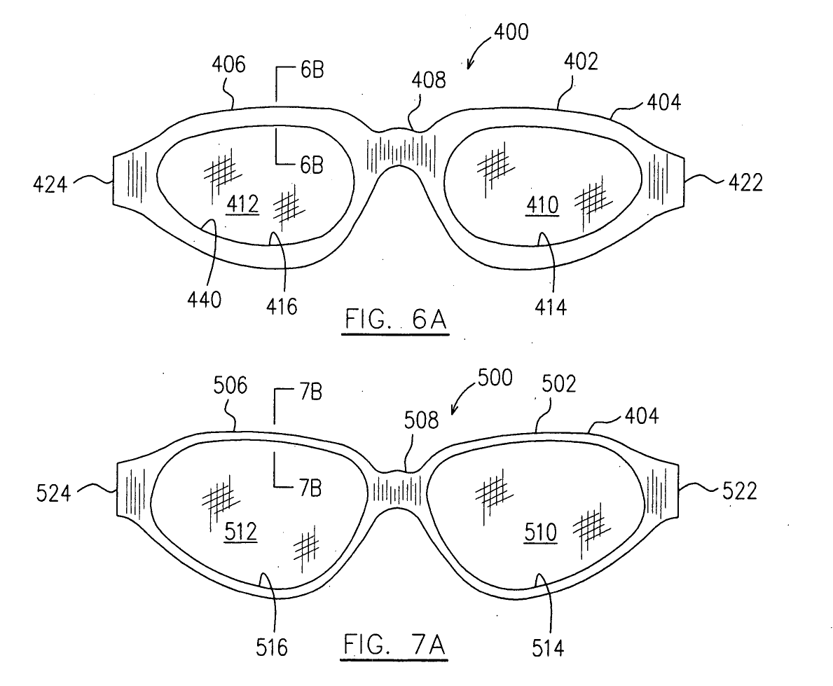

FIGS. 5 and 6A-6E illustrate a fourth embodiment of the present invention in which goggles having ventilation, generally designated by reference numeral 400, includes a frame structure 402 having two frame bodies 404, 406, with a bridge 408 connected between an inner side of the frame bodies 404, 406, and also includes a pair of lenses 410 and 412 received and affixed in apertures 414 and 416 which are defined in the respective frame bodies 404, 406. The attachment of the lenses 410, 412 to the respective frame bodies 404, 406 will be further described hereinafter. A pair of temples 418 and 420 are pivotally mounted to opposed sides 422, 424 of the frame structure 402 by means of a hinge assembly 426 (only one shown) which is well known in the art and will not be described herein. A plurality of contacting ribs 428 are provided on the inner side at the free end of the temples 418, 420 for comfortably holding the goggles 400 on the user's head when the goggles 400 is worn. An aperture 430 is also provided through each of the temples 418, 420 at its free end for optionally attaching an elastic strap (not shown) for further securing the goggles 400 on the user's head when the goggles 400 is worn.

The frame structure 402 including the frame bodies 404, 406 and the bridge 408 therebetween and the pivotally attached temples 418, 420 are preferably made of a substantially rigid plastic material. The lenses 410 and 412 are made of glass or substantially rigid transparent plastic material.

Shielding members 432, 434 are attached to the respective frame bodies 404, 406 on their inner side. The shielding members 432, 434 protrude from the inner side of the respective frame bodies 404, 406 and extend around the respective lenses 410, 412. Each of the shielding members 432, 434 has a contacting surface 436 which is contoured for comfortably contacting a portion of the face around the eye of the user when the goggles 400 is worn. Thus, each of the shielding members 432, 434 forms a substantial enclosure between one of the lenses 410, 412 and the face of the user when the contacting surface 436 of the shielding member 432, 434 contacts the face of the user. Ventilation channels 438 are formed on the contacting surface 436 of the respective shielding members 432 and 434 and are positioned in both the top section and bottom sections as well as in the outside section of the respective shielding members 432, 434, in order to ensure free passage of air from the enclosure to the atmosphere when the user wears the goggles 400. Thus, air flow around the eyes of the user eliminates or reduces moisture condensation on the respective lenses 410, 412. The shielding members 432, 434 are preferably made of flexible and soft material to be attached to the respective frame bodies 404 and 406. Alternatively, the shielding members 432, 434 can also be an integral part of the frame structure 402 and made of the same substantially rigid plastic material.

The goggles 400 is symmetrical about its central axis (not shown) and the left and right halves are identical. Therefore the description of a structural arrangement and method for permanent attachment of the lenses to the frame structure of the goggles will be simplified by making reference to only one lens attachment to the one frame body of the frame structure of the goggles in this embodiment, and also in the description of other embodiments described hereinafter.

In order to attach the lens 412 in the aperture 416 defined in the frame body 406, the frame body 406 includes a continuous front side wall 440 extending into the aperture 416 and contacting the peripheral portion on the front side of the lens 412. A projecting member 442 is formed integrally with the front side wall 440 and is filled into a hollow space defined in the peripheral portion on the front side of the lens 412. The hollow space defined in the lens 412 is a groove 444 extending along the peripheral portion of the lens 412. The groove 444 includes opposed side walls 446, 448 and a bottom 450. A distance W1 defined between the opposed walls 446, 448 at the opening (not indicated) of the groove 444 must be smaller than a distance W2 defined between the opposed walls 446, 448 at the bottom 450 of the groove 444. The projecting member 442 filled into the groove 444 during a formation molding procedure of the frame formation, is configured in accordance with the cross-section of the groove 444, thereby permanently engaging the lens 412 in the aperture 416 defined in the frame body 406.

The groove 444 may not be necessarily continuous along the entire periphery of the lens 412, as shown in FIG. 6D. Optionally, several sections of groove, for example 444a, 444b, 444c as shown in FIG. 6E can be provided in the lens 412. In order to cut a groove such as 444a, 444b or 444c in the lens 412 having W1 smaller than W2, the opening size W1 may be enlarged at one or both ends of the groove. 444a, 444b or 444c for entering and withdrawing a cutter, thereby forming an enlarged recess at one or both ends of the groove as shown in FIG. 6E. The continuous groove 444 as shown in FIG. 6D, however, needs only one enlarged recess at any position along the length of the groove for entering and withdrawing a cutter.

FIGS. 7A-7B illustrate a further embodiment of the present invention, in which an goggles having ventilation, generally designated by numeral reference 500, includes components similar to those of the goggles 400 of FIGS. 5 and 6A-6E. Components of the goggles 500 are indicated by numerals in the 500 series with the last two digits similar to the numerals indicating similar components of the goggles 400 of FIGS. 5 and 6A-6E, and will not therefore be redundantly described. The difference between the goggles 500 and the goggles 400 of FIGS. 5 and 6A is that instead of having the continuous front side wall 440 of FIGS. 6A and 6B, frame body 506 of FIGS. 7A and 7B includes a continuous rear side wall 541 extending into an aperture 516 and contacting the peripheral portion on the rear side of a lens 512. A projecting member 542 is formed integrally with the rear side wall 541 and is filled into a hollow space defined in the peripheral portion on the rear side of the lens 512. The hollow space defined in the lens 512 is a groove which can extend along the entire peripheral portion of the lens 512 or can include several sections similar to those illustrated in FIGS. 6D and 6E. The groove 544 has a cross-section which may be similar to the cross-section of the groove 444 as shown in FIG. 6C or may be configured differently, provided that the distance W1 is smaller than the distance W2. For example, in this embodiment, a T-shaped cross-section of the groove 544 is illustrated as an alternative example.

The structural arrangement of the goggles 400 of FIG. 6A and the goggles 500 of FIG. 7A are in fact similar. Nevertheless, by having different orientations of the structural arrangements, goggles 400 and goggles 500 not only provide different styling appearances but also affect the structural features of the respective shielding members 434, 534, as more clearly shown in FIGS. 6B and 7B. Frame body 406 having a continuous front side wall 440 provides a relatively narrow base for the shielding member 434 in contrast to the frame body 506 which includes a continuous rear side wall 541 and thereby provides a relatively large base for the shielding member 534. Those different structural features of the shielding members may be preferred in different types of goggles. For example, the frame body 506 may be preferred for attaching a soft shielding member 534 made of material such as sponge-type material. The frame body 406 having a relatively small base for the shielding member 434 may be preferred when the shielding member 434 is made of a substantially rigid plastic material and integrated with the frame body 406.

The formation of the frame bodies 406 and 506 with attachment of the respective lenses 412, 512 thereto is a molding procedure using a molding device similar to that described in FIG. 2C, and will not be repeated herein. Nevertherless, each of goggles 400 and 500 includes a pair of frame bodies and a bridge interconnecting same. Therefore, the molding device used to form the frame structure should have a cavity accordingly and thereby form the pair of frame bodies and the bridge simultaneously with permanent attachment of the pair lenses thereto.

It should be noted that other structural arrangements such as those illustrated in FIGS. 2B, 2D, 3B, 3C, 4B and 4C are also applicable to the goggles illustrated in FIG. 6A or FIG. 7A. Alternatively, the structural arrangements illustrated in FIGS. 6B and 7B are also applicable to goggles illustrated in FIG. 4.

The present invention is not limited to the examples described and illustrated above, and can be applied to other types of goggles having ventilation, for example those including a frame body having only an upper portion of the frame body which does not define a complete aperture for receiving the lens. With such a frame body, structural arrangements such as those illustrated in FIGS. 2E, 3C, 4B, 4C, 6B and 7B can be used for permanent attachment of a lens thereto.

The variety of structural arrangements of the present invention also advantageously provide a selection of styling options for goggles. For example, the embodiments illustrated in FIGS. 2A and 4A present novel styling features which cannot be achieved by conventional goggles using a “click-in” attachment.

It should be further noted that although permanent attachment of lenses to frame bodies is advantageously achieved especially when the lenses and the frame bodies are both are substantially rigid, the principle of the present invention is also applicable to attachment of lenses to frame bodies of goggles which do not require fluid-tight attachment of the lenses thereto and do not require fluid-tight seals around eyes of users, but are made of materials which may provide flexible or non-rigid features.

Modifications and improvements to the above-described embodiments of the present invention may become apparent to those skilled in the art. The foregoing description is intended to be exemplary rather than limiting. The scope of the present invention is therefore intended to be limited solely by the scope of the appended claims.

1. A method for permanently attaching a substantially rigid lens to a substantially rigid plastic frame of goggles having ventilation, the frame including a shielding member contoured for shielding the eyes of a user, the method comprising steps of:
providing a molding device for molding the substantially rigid plastic frame;
placing the lens in a predetermined position with respect to the molding device, in which position a portion of the molding device overlaps a portion of the lens; and
forming the substantially rigid plastic frame with permanent engagement of the lens thereto using the molding device.

2. A method as claimed in claim 1 further comprising a step of providing a hollow space defined in a portion of the lens to be attached such that a portion of the substantially rigid plastic frame is filled into the hollow space defined in the lens during the frame forming step, thereby permanently engaging the lens.

3. A method as claimed in claim 2 wherein the hollow space in the lens is defined as a plurality of holes extending through the portion thereof which the portion of the molding device overlaps such that a plurality of studs formed together with the plastic frame in the forming step, extend through the individual holes in each lens to permanently engage the lens to the substantially rigid plastic frame.

4. A method as claimed in claim 2 wherein the hollow space in the lens is defined as a groove formed in the portion thereof which the portion of the molding device overlaps, a distance between opposed walls of the groove at an opening thereof being smaller than a distance between the opposed walls at a bottom of the groove such that a projecting member formed together with the substantially rigid plastic frame in the forming step, is filled into the groove in each lens to permanently engage each lens the lens to the substantially rigid plastic frame.

5. A method as claimed in claim 1 wherein a continuous portion of the molding device overlaps a continuous peripheral portion of the lens at both sides thereof such that the substantially rigid plastic frame is formed with opposed continuous walls, thereby defining a continuous channel between the opposed continuous walls configured to correspond to the peripheral portion of the lens, the continuous channel having a depth to permanently engage the annular peripheral portion of each lens.

6. A structural arrangement for attaching a lens to a frame of goggles having ventilation, the frame including a shielding member contoured for shielding the eyes of a user, the structural arrangement comprising:
a frame body made of a plastic material, the frame body defining an aperture receiving the lens therein and defining a plurality of ventilation passages for circulating air between the face of the user and the lens;
a plurality of retaining members integrally formed together with the frame body; and
the respective retaining members being configured to achieve permanent attachment of the lens to the plastic frame body during formation of the frame body.

7. A structural arrangement as claimed in claim 6 further comprising:
a hollow space defined in the lens; and
a portion of the frame body filled into the hollow space of the lens during formation of the frame body, thereby permanently engaging the lens.

8. A structural arrangement as claimed in claim 7 wherein the retaining members comprise first and second groups thereof, the respective first and second groups of the retaining members abutting opposed sides of the lens and having a plurality of studs extending between the respective first and second groups of the retaining members and crossing the lens through a plurality of holes defined as the hollow space in the lens, the studs being formed integrally with the respective first and second groups of the retaining members during the formation of the frame body.

9. A structural arrangement as claimed in claim 7 wherein the hollow space is defined as a plurality of holes extending through the lens and wherein the portion of the frame body filled into the hollow space is defined as a plurality of studs integrated with the frame body and extending through the respective holes.

10. A structural arrangement as claimed in claim 9 wherein each stud extending from the frame body through one of the holes in the lens comprises an enlarged end having a size greater than a diameter of the hole in order to permanently engage the lens between the frame body and the enlarged end thereof.

11. A structural arrangement as claimed in claim 7 wherein the hollow space in the lens is defined as a groove formed in the lens at one side thereof, a distance between opposed walls of the groove at an opening thereof being smaller than a distance between the opposed walls at a bottom of the groove such that a projecting member formed together with the frame body during the formation of the frame body, is filled into the groove in the lens to permanently engage the lens to the frame body.

12. A structural arrangement for attaching a substantially rigid lens to a frame of goggles, the frame including a shielding member contoured for shielding the eyes of a user, the structural arrangement comprising:
a frame body made of a substantially rigid plastic material;
a plurality of retaining members integrally formed together with the substantially rigid plastic frame body; and
the respective retaining members being configured to achieve permanent attachment of the lens to the substantially rigid plastic frame body during formation of the frame body.

13. A structural arrangement as claimed in claim 12 wherein the retaining members comprise opposed continuous side walls extending into an aperture receiving the lens, thereby defining a continuous channel therebetween, the continuous channel being configured to correspond to a periphery of the lens for receiving the periphery of the lens therein, and having a depth sufficient to permanently engage the lens.

14. A structural arrangement as claimed in claim 13 wherein the frame body comprises a plurality of studs disposed in the continuous channel, each stud extending between the opposed side walls of the channel and crossing the lens through one of a plurality of holes defined in the lens.

15. A structural arrangement as claimed in claim 12 wherein the lens comprises a hollow space defined therein, and wherein the retaining members comprise a portion of the frame body filled into the hollow space of the lens during formation of the substantially rigid plastic frame body, thereby permanently engaging the lens.

16. A structural arrangement as claimed in claim 1-5 wherein the hollow space is defined as a plurality of holes extending through the lens and wherein the portion of the substantially rigid plastic frame body filled into the hollow space is defined as a plurality of studs integrated with the frame body and extending through the respective holes.

17. A structural arrangement as claimed in claim 16 wherein each stud extending from the substantially rigid plastic frame body through one of the holes in the lens comprises an enlarged end having a greater than a diameter of the hole in order to permanently engage the lens between the frame body and the enlarged end thereof.

18. A structural arrangement as claimed in claim 15 wherein the hollow space in the lens is defined as a groove formed in the lens at one side thereof, a distance between opposed walls of the groove at an opening thereof being smaller than a distance between the opposed walls at a bottom of the groove such that a projecting member formed together with the frame body during the formation of the substantially rigid plastic frame body, is filled into the groove in the lens to permanently engage the lens.
